(19) United States
(12) Patent Application Publication
(10) Pub. No.: US 2012/0245518 A1
(43) Pub. Date: Sep. 27, 2012

(54) PHARMACEUTICAL COMPOSITION FOR THE TREATMENT OF BLADDER DISORDERS

(75) Inventors: Sándor Lovász, Budapest (HU); Beáta Furka, Budapest (HU); Ákosné Székely, Budapest (HU); Gáborné Forrai, Budapest (HU); Maria Rodenné Juhász, Budapest (HU); Lajos Sényi, Budapest (HU)

(73) Assignee: Ritcher Gedeon NYRT., Budapest (HU)

(21) Appl. No.: 13/499,385

(22) PCT Filed: Nov. 18, 2010
(86) PCT No.: PCT/HU10/00125

§ 371 Date: Jun. 5, 2012

(30) Foreign Application Priority Data
Nov. 18, 2009 (HU) P09 00717

(51) Int. Cl. A61M 29/00; A61K 31/728; A61P 13/10; C08B 37/08

(52) U.S. Cl. 604/96.01; 536/55.1; 514/54

(57) ABSTRACT

The invention relates to pharmaceutical compositions with activity against disorders of urogenital system of mammals which comprises a zinc-hyaluronan complex as active ingredient and a pharmaceutically acceptable carrier and/or additive. The process for the preparation of said pharmaceutical compositions as well as the therapeutic use thereof for the treatment and prevention of diseases associated with abnormalities and deficiencies of glucosaminoglycan (GAG) layer in the urogenital system in mammals and a kit comprising zinc hyaluronate solution, a catheter which is applicable to intravesical administration and optionally a balloon which is applicable to bladder dillatation are also within the scope of the invention.

### FIELD OF THE INVENTION

The invention relates to pharmaceutical compositions with activity against disorders of urogenital system of mammals which comprises a zinc-hyaluronan complex as active ingredient and a pharmaceutically acceptable carrier and/or additive. The process for the preparation of said pharmaceutical compositions as well as the therapeutic use thereof for the treatment and prevention of diseases associated with abnormalities and deficiencies of glucosaminoglycan (GAG) layer in the urogenital system in mammals and a kit comprising zinc hyaluronate solution, a catheter which is applicable to intravesical administration and optionally a balloon which is applicable to bladder dillatation are also within the scope of the invention.

### BACKGROUND OF THE INVENTION

The hyaluronan (HA) is a homopolymer of glucosaminoglycan type built up of repeated N-acetylglucosamin-glucuronic acid disaccharide units of the formula (I).

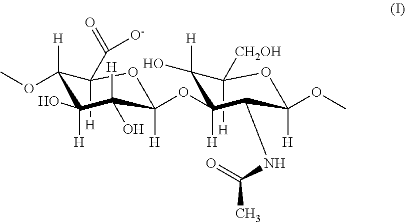

In the HA the monosaccharides are β(1→3) linked, whereas the disaccharide units are β(1→4) linked, thus forming a linear polysaccharide with alternating β(1→3) and β(1→4) linkages.

The HA in living organisms occurs as a salt formed with a cation, usually sodium, and its molecular weight may range from 10-20 kDa to several thousands kDa. The presence of carboxyl group in the glucuronic acid moiety of the HA and the carbonyl and amino groups in the N-acetyl group of the glucosamine, as well as the hydroxyl groups being present facilitate the formation of several hydrogen bridges. Due to these intramolecular hydrogen bonds and the hydrogen bridges formed via interactions between HA and the water being present in the biological systems, HA has a complicated three dimensional structure (C. L. Hew et al., Eur. J. Biochem. 203, 33-42. (1992); Q. Liu et al. J. Am. Chem. Soc 118, 12276-12286. (1996)). As a consequence of its exceptional water binding ability, even relatively dilute aqueous solutions of HA show high viscosity. In aqueous solutions its rheologic properties are highly dependent upon the size of the molecule; e.g. in 1% aqueous solution HA of the 1000 kDa molecular size has 3000 mPa viscosity, whereas HA of the 4000 kDa molecular size has 400000 mPa viscosity in similar concentration (H. B. Wik and O. Wik: Rheology of Hialuronan, The Chemistry, Biology and Medical Applications of Hialuronan and its Derivatives (ed. T. C. Laurent) pp. 25-32. Portland Press, London, (1998)). Accordingly, the two most important physical features of HA are the viscosity and the molecular size thereof.

As a main component of the extracellular matrix HA is present in all parts of the body. Certain organs and tissues (connective tissues, skin, synovial fluid, vitreous humour, and blood vessel wall) contain HA in an increased amount. It has long been thought that the biological role of HA derived from its physical properties. For instance, it can provide mechanical protection to joints by virtue of its rheological nature. Owing to its exceptional water binding ability, HA can control the water balance through its osmotic pressure and by offering resistance to flow. HA also plays an important role in filling up the interstitium and protects cells from different physical impacts. Recent investigations showed that the interaction between HA and certain macromolecules present in the body could be brought into connection with several physiological processes. Examples of such macromolecules are the proteoglycanes (aggrecan, versican, brevican, etc.) which are situated in the extracellular matrix and have the main task to occupy the space between cells and to facilitate material transport. Macromolecules entering into interactions with HA can be intracellular transmembrane proteins (CD44, RHAMM), as well as receptor proteins present in the cytoplasm (C1q, P-32, TSG-6, etc.). Through the proteins mentioned above, HA plays an important role in several control processes taking place at cell or body level.

The molecular size and the concentration of HA in aqueous solution have—as it is the case with the physical properties—a considerable influence on its biological effect. Thus, depending on the size of the molecule, HA may exert either a positive or a negative effect on the same cellular process. Similar change of effect was seen when the HA concentration of the solution had been altered. The desired optimal effect can obviously be achieved when both parameters are simultaneously taken into consideration (E. A. Balázs, The Chemistry, Biology and Medical Applications of Hialuronan and its Derivatives (ed. T. C. Laurent) pp. 185-204. Portland Press, London (1998)).

Since HA participates in the physiological processes mentioned above, it can successfully be used in several fields of therapy (wound-healing, treatment of chronic inflammation, ophthalmic surgery).

The range within which HA is applicable in the human therapy—beyond those mentioned above—can be widened by modifying the structure chemically. In this respect, two main trends are known. According to one of them, cross-linkages are established between two distant positions of the HA molecule by using an aliphatic compound (usually a dihydrazide) to form a hydrogel. The cross-linkages cause an increase in the viscoelasticity of the chemically modified HA resulting in greater resistance to degradation effects occurring in the body. It is to the advantage of patients with rheumatoid arthritis treated to regain the synovial fluid or of those having postoperative adhesion. In another important strategy active agents which are difficult to absorb or are to be passed specifically to the location of effect are chemically bound to the HA (e.g. taxol, pilocarpine, insulin). In these cases HA causes improved absorption of the active agents bound to HA and assists specific arrival of the matter to the target place, respectively.

Complexes of HA formed with zinc and cobalt are described in EP 413016 (Burger et al., 1989), and of the two the Zinc-HA is used as active agent in compositions for wound treatment. Further studies of the active agent showed that due to the presence of zinc possibilities for therapeutic use can be broadened, since the zinc complex has new or more expressed effects compared with the sodium-HA salt (J. Illés et al., Acta Pharm. Hung. 72, 15-24 (2002)). Among others, such effects are the increased antioxidant activity of the zinc-HA (Gy. T. Balogh et al., Arch. Biochem. Biophys. 410, 76-82. (2003)); the inhibitory effect on tissue damaging enzymes (matrix metalloproteinases, particularly the MMP-9) which are produced in increased amount by invasive cells, while the sodium-HA salt does not show the latter effect (WO 00/53194, Illés et al. 1999). The gastroprotective effect (treatment of peptic ulcer) of the zinc-HA is disclosed in published WO 98/48815 international patent application, antimicrobial effect of the zinc-HA active agent is described in published WO 98/10773 international patent application.

A glycosaminoglycan (GAG) layer which contains a large amount of hyaluronic acid lines the inner surface of the urinary bladder. This highly viscous, highly hydrophilic GAG layer protects the bladder epithelium against irritants in the urine including, but not limited to, microorganisms, pathogens, microcrystals, proteins, calcium, urea and carcinogens.

When the GAG layer is damaged the bladder epithelium becomes permeable to urinary irritants. This causes immediate pain and this is the beginning of a degradation process as well. The damage of this protective barrier increases the risk for development of infections and tumorous defects. The deformation of the GAG layer can be proved by biopsy.

The following disease can be associated with the lesion of the GAG layer:
Interstitial cystitis
Chronic recurrent bacterial and non-bacterial cystitis
Radiation cystitis and chemical cystitis

Interstitial cystitis is a condition that results recurring discomfort or pain in the bladder and surrounding pelvis region. The symptoms also include urgency for urination and increased frequency of urination. At the same time, bacterial cannot be proved.

Individuals with interstitial cystitis can be significantly disabled, and individuals with advanced interstitial cystitis may require major surgery in order to function. Although the etiology of interstitial cystitis remains unexplained, it has been suggested that deficiencies in the GAG layer may be a primary defect. Because the symptoms are similar to those of other disorders of the urinary system, the diagnosis of interstitial cystitis can be very difficult and in most cases takes a long time (5-8 years).

Radiation cystitis and chemical cystitis may emerge after radiation and chemical therapy of tumors in the pelvic organs (prostate, urinary bladder, uterus, ovary, vagina, colon, rectum). One of the main effect of radiotherapy is the damage of the bladder epithelium. The duration of the disease is usually 3 to 6 months, but it can be 24 months or longer. In the most serious cases lifestyle altering symptoms can emerge. Without an adequate therapy, the inflammation in the bladder may become more serious and the symptoms can worsen.

Recurrent bacterial cystitis is defined as recurrant significant bacteriurea, in the absence of upper tract patology. “Recurrant” is usually taken to mean more than three proven bacterial cystitis in a year. Acute bacterial cystitis is a very common infection among sexually active women. It will be experienced by 30 to 50% of females in their lives and it may become reccurent 25 to 40% of them. The common treatment is a longlasting antibiotic therapy. In the case of urethal-syndrome no bacteria in the urine can be found. In both cases the damage of the GAG layer is probable.

Sodium hyaluronate is recently used in the treatment of diseases of the urogenital system especially in the treatment of interstitial cystitis and radiation cystitis administered intravesically using uretheral catheter (Binoche WO 96/25168 and WOO 00/24387). WO 96/25168 describes the use of sodium hyaluronate in the treatment of interstitial cystitis. Efficacy of intravesically administered sodium hyaluronate solution was proved by clinical experiments. Outcome criteria for the study were related to improvement of symptoms based on decreases in pain and urgency. The patients received intravesical intstillations of sodium hyaluronate once per week for 4 weeks, it was followed by maintanance instillation once every 4 week for 20 week-period. Every time the patients received intravesical instillation of 50 ml of 0.08% sodium hyaluronate solution. HA composition was instilled into the bladder using an uretheral catheter. Then the patient retained the HA solution for a minimum of 30 minutes. 14 patients were observed. At the end of the study, 5 of them reported considerable improvement in their symptoms. The others did not feel any improvement or just a little.

### SUMMARY OF THE INVENTION

The invention relates to pharmaceutical compositions with activity against disorders of urogenital system of mammals which comprises a zinc-hyaluronan complex as active ingredient and a pharmaceutically acceptable carrier and/or additive. The process for the preparation of said pharmaceutical compositions as well as the therapeutic use thereof for the treatment and prevention of diseases associated with abnormalities and deficiencies of glucosaminoglycan (GAG) layer of the urogenital system in mammals are also within the scope of the invention.

### BRIEF DESCRIPTION OF FIGURES

FIG. 1: Equipment with balloon catheter suitable for administration of an active ingridient under pressure

FIG. 2: Scores on visual analog pain scale and daily average urine portions of the first patient (Example 5) as a function of time

FIG. 3: Scores on visual analog pain scale and daily average urine portions of the second patient (Example 5) as a function of time

FIG. 4: A balloon catheter which is applicable to bladder dilatation

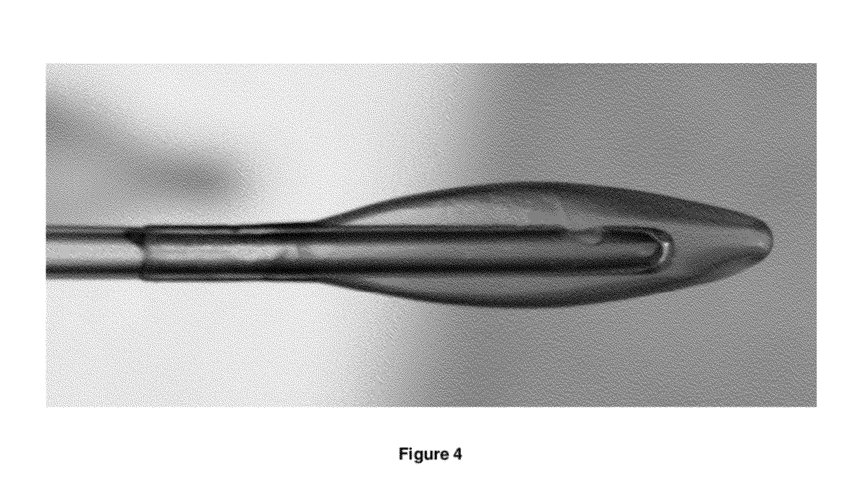

### DETAILED DESCRIPTION OF THE INVENTION

The invention relates to the use of zinc hyaluronate complex in the manufacture of a medicament for the treatment and prevention of diseases associated with abnormalities and deficiencies of glucosaminoglycan (GAG) layer of the urogenital system in mammals.

We have found that the use of zinc hyaluronate in intravesical therapy not only regenerates the GAG layer but also induces improvement in the deeper layers of bladder epithelium.

According to our studies, we have found that before the use of zinc hyaluronate the treatment of bacterial infection was not necessary. The signs of regeneration of the GAG layer could be observed on biopsy samples despite of the uncured bacterial infection.

The above mentioned observations are surprising because prior to sodium hyaluronate therapy the treatment of bacterial infection is necessary since the toxins and harmful metabolites of bacteria in urine may inhibit the regeneration process. A combined therapy of zinc hyaluronate solution and an antibiotic may have synergistic effect, it may reduce the length of therapy and may extend the length of remission.

In one desired embodiment of the present invention, the zinc hyaluronate solution is administered intravesically.

In other desired embodiment, the intravesical administration of zinc hyaluronate solution is combined with balloon dilatation of the bladder, especially in the case of patients suffering from interstitial cystitis. Zinc hyaluronate solution is administered via catheter into the bladder, then hydrodilatation of bladder is performed with a balloon.

The symptoms of interstitial cystitis include the decrease of bladder capacity. As the disease develops the elasticity of the bladder wall is getting worse. Hydrodilatation of the bladder is a common method in urology. The method according to the present invention includes the administration of zinc hyaluronate solution combined with the dilatation of the bladder. The zinc hyaluronate solution in the bladder get under pressure after the balloon is filled up. The solution remains between the bladder wall and the balloon. The pressure in the bladder inhibits the urine from getting into the bladder, and so the concentration of the solution doesn't decrease. Further advantages of this method are that the surface of the bladder wall extends, the mucosa gets thiner and the active ingredient can diffuse into the deeper layers of the mucosa. During the treatment a catheter is inserted into the bladder and after the drainage of the residual urine, the solution of the active ingredient is introduced into the bladder via the same catheter. Then the catheter is removed and a balloon, which is applicable to bladder dilatation is inserted into the bladder. The filled-up balloon helps the solution of the active ingredient to disperse consistently on the bladder wall.

The balloon catheter according to the present invention which is applicable to bladder dilatation, may comprise a plastic catheter and a thin, spherical (when filled-up) advantegously 5 cm long balloon at the end of the catheter.

The pharmaceutical composition of the present invention is advantegously a solution and concentration of zinc hyaluronate is 0.01-5 mg/ml. The solution of zinc hyaluronate can be prepared in several ways.

The solution may be prepared by solving solid zinc hyaluronate in sterile water. Other agents (isotonic, preservative agents) may also be added to the solution.

The solution may also be prepared in situ from aqueous solution of sodium hyaluronate.

When an appropriate zinc compound is added to aqueous solution of sodium hyaluronate, the zinc ions displace the sodium ions and zinc hyaluronate solution occurs. The above mentioned agents may also be added to the solution.

The pharmaceutical composition according to the invention contains the zinc hyaluronate complex active ingredient in a suitable liquid carrier e.g. in a sterile aqueous solvent. It may also contain water soluble isotonic agents such as sodium chloride or sorbit, and other agents, e.g. presevative agents such as sodium bisulfite, sodium bisulfate, sodium thiosulfate, potassium sorbate, methylparaben, polyvinyl alcohol, phenyl ethyl alcohol and buffering agents such as sodium carbonate, sodium borate, sodium phosphate, sodium acetate, sodium bicarbonate.

The concentration of the above mentioned non-active agents in the solution may be varied between 0.001 and 5 mass percent.

### EXAMPLES

The following example is merely illustrative of the present invention and should not be construed as limiting the scope of the invention in any way as many variations and equivalents that are encompassed by the present invention will become apparent to those skilled in the art upon reading the present disclosure.

### Example 1

After weighing 0.20 mg sodium hyaluronate (Reag. Ph. Eur.) in a 100 ml flask, 5.0 ml of zinc chloride solution of 0.10 mol/litre concentration prepared with twice distilled water (water for injection use, pyrogen-free, sterile) are added, then the volume is filled up to 50 ml twice distilled water. Then 23.5 ml of sorbitol solution of 1.00 mol/litre concentration is added.

(The solution is prepared with twice distilled water.) Subsequently, the volume is filled up to 100 ml with twice distilled water. Finally, the solution is filtered off trough a membrane filter.

### Animal Model Studies

Two series of experiments were designed to evaluate the efficacy of zinc hyaluronate solution in the regeneration of the bladder wall. In the first series an experimental pathological process served as a model of the alterations that develop in the bladder wall in interstitial cystitis. In the second series of experiments an acute inflammatory process of the bladder was modeled.

### Example 2

### Model of Interstitial Cystitis

Experiments were performed on 20 white female rats. An experimental pathological process similar to that occurring in the bladder wall in interstitial cystitis has been modelled. Cryodestruction was caused in the bladder. A tampon impregnated with liquid nitrogen was introduced into bladder and retained for 20 seconds to induce interstitial cystitis. Then, animals were selected into three groups.
Group 1: rats have been treated only once after 48 hours following cryodestruction, 1 ml of zinc hyaluronate solution (according to example 1) was introduced into the bladder and retained for 30 minutes.
Group 2: animals received the same treatment as the animals in the first group but three times (on 3 consecutive days)
Group 3: 3 control animals received no treatment after cryodestruction

Then, rats were killed with an overdose of sodium thiopental and the bladders were excised. For histological studies, the samples were fixed in 10% buffered formalin and treated with standard technique. The samples were stained with toluidine blue. Toluidine blue can stain dameged mucosa well, metachromasy depends on the degree of the damage in the tissue. Histological study on the control animals showed mucosal ulceration, lymphohistiocyte infiltration with polymorphonuclear leukocytes in the perifocal area, and pronounced vasodilatation of the microcirculatory vessels. Samples stained with toluidine blue revealed metachromasy of the connective tissue. Electron-microscopic studies showed destructon of the collagen fibers with loss of their striated structure. The above morphological changes correspond to the picture of interstitial cystitis.

According to histological studies of the treated animals, the most pronounced beneficial changes developed in the connective tissue. These changes were significant after a single treatment with zinc hyaluronate solution. Administration of zinc hyaluronate solution on three occasions further developed the changes in the connective tissue.

Electron-microscopic findings in the case of single treated animals exhibit stabilization of the collagen fibers and partial restitution of their striated structure. After the triple treatment, the stabilization of the collagen fibers was more significant and restitution of their striated structure was complete.

### Example 3

### Acute Bacterial Cystitis Model

Experiments were performed on 16 white female rats. An acute inflammation of the bladder was induced by injection (under pressure) of 1.0 ml of E. Coli culture (10⁶ CFU/ml).

Then, animals were selected into three groups:
Group 1: rats have been treated only once after 48 hours following E. coli injection, 1 ml of zinc hyaluronate solution (according to example 1) was introduced into the bladder and retained for 30 minutes.
Group 2: animals received the same treatment as the animals in the first group but three times (on 3 consecutive days)
Group 3: 3 control animals received no treatment after cryodestruction

Then, rats were killed with an overdose of sodium thiopental and the bladders were excised. For histological studies the samples were fixed in 10% buffered formalin and treated with standard technique. The samples were stained with toluidine blue.

Histological study on the control animals showed the following changes:
Marked infiltration of all layers of the bladder wall
Vasodilation of the microcirculatory vessels
Edema
Focal destruction of the bladder epithelium and its connective tissue

Microscopic study of the toluidine blue stained samples showed significant metachromasy in the connective tissue.

Electron-microscopic findings in the case of treated animals verified that zinc hyaluronate treatment markedly stabilized collagen fibers, decreased edema and diminished metachromasia, indicating stabilization of glucosaminoglycans.

Zn hyaluronate treatment also beneficially affected epithelization process in both models. After one-dose treatment, the expression of the edge epithelization was 14.35% higher than in the controll animals. Repeated treatment further increased the difference to 21.51%.

### Clinical Study

Inclusion criteria for this study include:
1. female
2. >18 years of age
3. urgency for urination and increased frequency of urination or pelvic pain for at least 6 months
4. urination for at least 7 times a day or stimulus to urinate or pelvic pain (measured on Visual Analog Scale)
5. Hydrodistension under anesthesia to 60 to 80 cm H2O pressure with glomerulations and bloody effluent
6. sterile urine cultures

Exclusion criteria for this study include:
1. Average amount of urine is more than 150 ml
2. tuberculosis of urogenital system
3. benign or malignant bladder tumors
4. patients taking any medication or active treatment for interstitial cystitis treatment within 30 days of enrollment in study
5. patients taking any medication which effects bladder within 1 week
6. pregnancy, lactation
7. ovarian, cervical and vaginal cancer
8. vaginal infections
9. bacterial cystitis within 3 months
10. active herpes within 3 months
11. cyclophosamide treatment in anamnesis
12. Radiation cystitis
13. neurogenic bladder dysfunction
14. bladder inflammation within 3 months
15. surgery for bladder dilatation or excision

The effects of zinc hyaluronate treatment are assassed using visual analog pain scale and measuring the amount of urine portions.

### Example 4

### Administration of Zinc Hyaluronate Solution Intravesically

8 patients were treated.

A bladder catheter was inserted into the bladder, the residual urine in the bladder was drained, then 10 ml of zinc hyaluronate solution (according to Example 1) was introduced into the bladder and retained for 1-1.5 hours. The treatment was repeated weekly for 3-5 weeks depending on patient's condition. Two patients reported no improvement in their symptoms. The other six patients reported improvement in urgency and frequency and in the urination independent symptoms such as pelvic pain as well few hours after the first treatment. It meant 2-3 scores decrease on the visual analog pain scale. Two of them reported further 2-3 scores decrease after following treatments, this improvement in symptoms lasted for 1-2 months. Four of them reported a 100% improvement in their symptoms. With once a month treatment their condition became permanent.

### Example 5

### Combination Therapy

### Administration of Zinc Hyaluronate Solution Intravesically and Bladder Dilatation

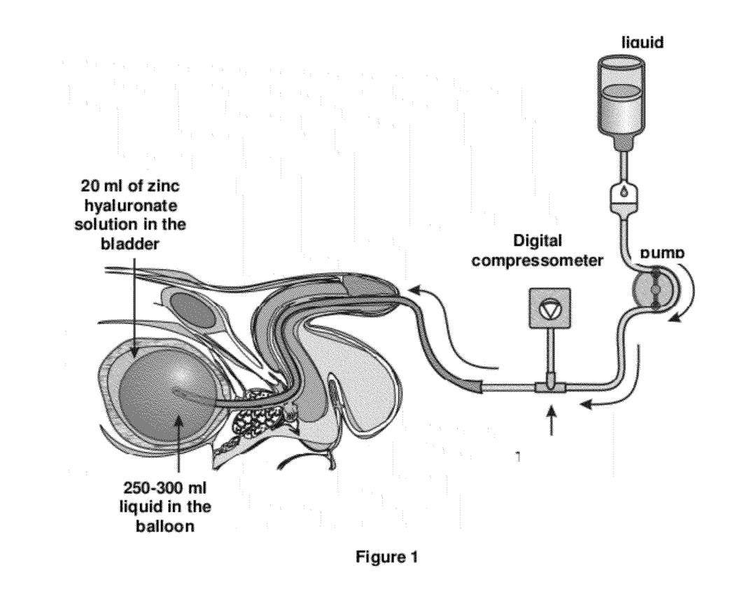

The administration of zinc hyaluronate was combined with the balloon dilatation of the bladder. After or before the treatment patients get zinc hyaluronate solution intravesically on several occasions according to the previous example. A bladder catheter was inserted into the bladder, the residual urine in the bladder was drained, then 20 ml of zinc hyaluronate solution (according to Example 1) was introduced into the bladder. Then, the catheter was removed and a balloon catheter was inserted into the bladder. Then the balloon was filled up with the equipment shown in FIG. 1.

After filling-up, the zinc hyaluronate solution in the badder got under pressure. Because of the expansion of the bladder the surface of it extends and active ingredient can diffuse passively into deeper layers of the mucosa. In order to avoid side effects (such as the injury of the bladder wall) the pressure of the bladder wall was measured trough the whole treatment. The pressure was increased to 80 H2O cm and retained for 10 minutes.

The above described therapy was performed on 10 patients. A few days after combination therapy, the patients reported 100% improvement in pain. 8-9 days after bladder dilatation the capacity of the bladder increased significantly and remained permanently.

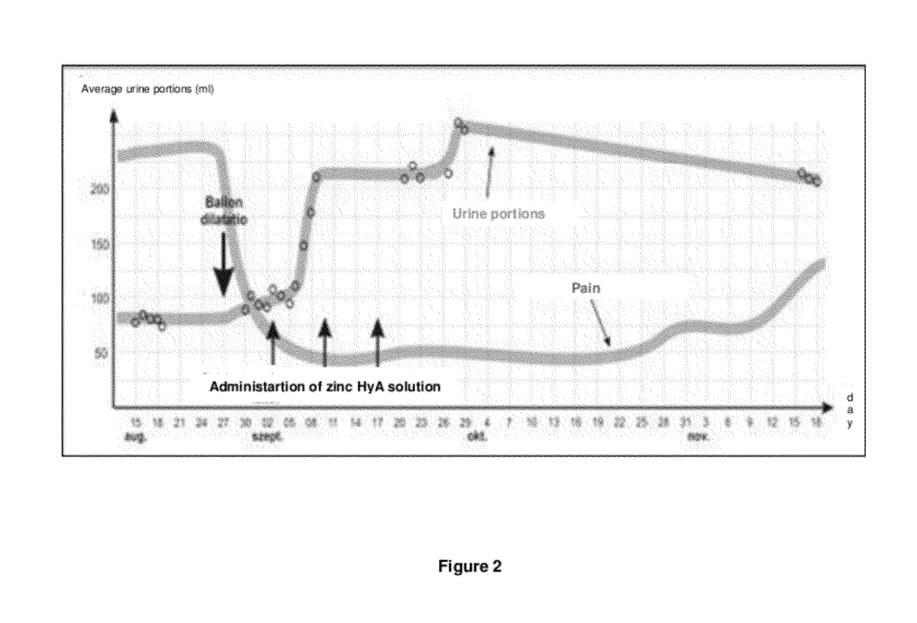

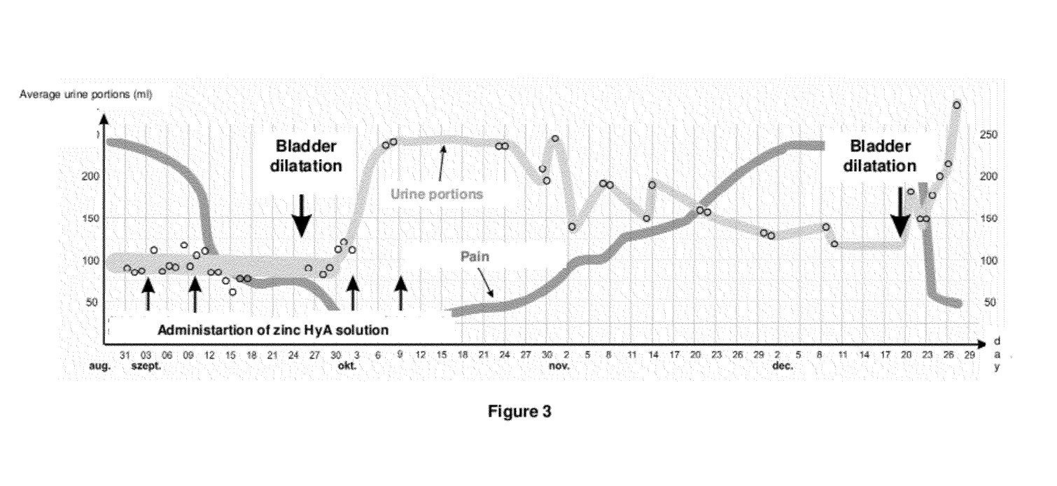

The improvement in two patients' symptoms are shown in FIGS. 2-3. One of the curves shows the impovement in pain, the other shows the change in the amount of urine portions. The date of the combined treatment and the administrations of zinc hyaluronate solution alone are assigned on the diagram. According to the diagram, it is clear that zinc hyaluronate therapy alone can decrease pain but cannot inscrease the capacity of the bladder. After 3 months, the second patient (FIG. 3) received a combination treatment again. We have found that the results could be repoduced.

### Treatment of Chemical Cystitis Induced by Intravesically Administered Cytostatic Medicament

### Example 6

The treatment was the same as that described in Example 5. Two patients were treated who had bladder excision surgery because of malignant tumor. After surgery, they received cytostatic medicament intravesically. Their symptoms after the cytostatic treatment were very similar to those of interstitial cystitis.

After the first treatment (described in Example 5) patients reported an almost 100% improvement in symptoms. The scores on Visual Analog Pain scale decreased by 80% and the amount of average urine portions increased by 40-50%.

The effects of the treatment remained permanent in the next 4-6 months.

1. A pharmaceutical composition comprising a zinc-hyaluronate complex for the treatment and prevention of diseases associated with abnormalities and deficiencies of glucosaminoglycan (GAG) layer of the urogenital system in mammals.

2. A pharmaceutical composition according to claim 1 for the treatment of cystitis.

3. A pharmaceutical composition according to claim 1 for the treatment of interstitial cystitis.

4. A pharmaceutical composition according to claim 1 in the form of a solution.

5. A pharmaceutical composition according to claim 4 wherein the concentration of the solution is 0.01-5 mg/ml.

6. A pharmaceutical composition according to claim 4 wherein the internal pressure of the solution is 50-80 H₂O cm.

7. The use of zinc hyaluronate complex in the manufacture of a medicament for the treatment and prevention of diseases associated with abnormalities and deficiencies of glucosaminoglycan (GAG) layer of the urogenital system in mammals.

8. (canceled)

9. (canceled)

10. A method of treating and preventing diseases associated with abnormalities and deficiencies of glucosaminoglycan (GAG) layer of the urogenital system in mammals said method comprising administering a pharmaceutical composition comprising a zinc-hyaluronate complex to a mammal.

11. The method of claim 10 wherein the composition is administered intravesically into the bladder optionally, followed by a balloon dilatation of the bladder.

12. The method of claim 10 wherein the pharmaceutical composition is in the form of a solution.

13. The method of claim 12 wherein the concentration of the solution is 0.01-5 mg/ml.

14. The method of claim 12 wherein the internal pressure of the solution is 50-80 H₂O cm.

15. A kit comprising the pharmaceutical composition according to claim 1, a catheter which is applicable to intravesical administration and optionally a balloon which is applicable to bladder dillatation.

16. A kit according to claim 15 characterized in that it comprises a balloon which is applicable to bladder dilatation.

17. The method of claim 10 wherein the mammal suffers from a disease associated with abnormalities and deficiencies of glucosaminoglycan (GAG) layer of the urogenital system.

18. The method of claim 17 for the treatment of cystitis.

19. The method of claim 17 for the treatment of interstitial cystitis.

20. The method of claim 17 wherein the composition is administered intravesically into the bladder followed by a balloon dilatation of the bladder.

21. The method of claim 20 wherein the pharmaceutical composition is in the form of a solution.

22. The method of claim 21 wherein the concentration of the solution is 0.01-5 mg/ml.
